(19) United States
(12) Patent Application Publication
(10) Pub. No.: US 2016/0303358 A1
(43) Pub. Date: Oct. 20, 2016

(54) System for combined vacuum and instillation treatment of wounds

(71) Applicant: PAUL HARTMANN AG, Heidenheim (DE)

(72) Inventors: Pierre Croizat, Herbrechtingen (DE); Axel Eckstein, Heidenheim (DE); Karina Beyrle, Langenau (DE)

(21) Appl. No.: 15/100,356

(22) PCT Filed: Dec. 8, 2014
(86) PCT No.: PCT/EP2014/076924

§ 371 Date: May 31, 2016

(30) Foreign Application Priority Data
Dec. 19, 2013 (DE) 102013226713.4

(51) Int. Cl. A61M 35/00; A61M 39/28; A61M 1/00

(52) U.S. Cl. CPC A61M 35/00; A61M 1/0088; A61M 1/0023; A61M 1/0084; A61M 39/28; A61M 2205/36; A61M 2205/3584; A61M 2205/505; A61M 2205/52

(57) ABSTRACT

A system (10) for combined vacuum and instillation treatment of wounds on the human or animal body. The system (10) has a vacuum unit (14) with a housing (18) of a first housing type and an instillation unit (30, 30a, 30b) with a housing (34, 34a 34b) of a second housing type. The housing (34, 34a, 34b) of the second housing type has an openable and closable cover (46, 46a, 46b) through which a conduit passage area (42) is accessible. The housing (18) of the first housing type has a first securing mechanism (50) which can be connected to a second securing mechanism (52, 52a, 52b) of the housing (34, 34a, 34b) of the second housing type with a form fit, a snap fit, a latching fit, a fit engaging behind with a form fit or with a clamping fit.

The invention relates to a system for combined vacuum and instillation treatment of wounds on the human or animal body, wherein the system comprises a vacuum unit with a housing of a first housing type, with a pump for providing a vacuum, and with a liquid collection container for receiving bodily fluids, in particular wound secretions aspirated from a wound, wherein the container can be secured releasably on the housing of the first housing type, and an instillation unit with a housing of a second housing type, with a conduit passage area for at least one inward conduit portion and an outward conduit portion, with a feed pump, preferably a peristaltic pump, wherein the housings of the first and second housing types are each housings that are separate from each other or separable from each other.

Vacuum therapy and instillation therapy are both used, for example, in the treatment of wounds that are difficult to heal. In the vacuum treatment of wounds, a pressure-tight cover is placed over the wound, and the space between cover and wound is exposed to a vacuum. This promotes wound healing. Instillation therapy signifies wound treatment in which fluid media, in particular liquid medicaments, are introduced into the wound. Examples of such liquid media are solutions, emulsions or suspensions of antibiotics, analgesics or local anesthetics. In this way, for example, inflammation that is present in the wound area and that impairs the wound-healing process can be treated or prevented.

The combined use of vacuum and instillation treatment brings together the advantages of both treatment methods. Within the meaning of the present invention, housings that are separate from each other or separable from each other are to be understood as housings that permit separate handling of the instillation unit and of the vacuum unit. The separate housings are in this case designed in such a way that the instillation unit and the vacuum unit represent functionally separate units, that is to say no components of the instillation unit are accommodated in the housing of the vacuum unit, and, conversely, no components of the vacuum unit are accommodated in the housing of the instillation unit. Excluded from this are control components in the sense of master/slave control.

The object of the present invention is to make available a system which simplifies the treatment of wounds by means of combined instillation and vacuum therapy and, in particular, improves the operation and handling of the devices used for this purpose.

In a system of the type mentioned at the outset, this object is achieved, according to the invention, in that the housing of the second housing type has an openable and closable cover through which the conduit passage area is accessible, and in that the housing of the first housing type has a first securing mechanism, and in that the housing of the second housing type has a second securing mechanism, wherein the first securing mechanism can be connected to the second securing mechanism with a form fit, preferably a snap fit, a latching fit, or some other form-fit engagement from behind, or a clamping fit. In this way, the vacuum unit and the instillation unit can be joined together to form one manageable device unit. A particularly advantageous design is one in which the two units can be joined together to form a device tower. The cover designed according to the invention permits in this case simple access to the conduit passage area in which the conduits of the instillation unit are arranged or can be arranged. It proves advantageous if the cover is mounted pivotably on the housing of the second housing type. In this way, the cover cannot accidentally come loose from the housing of the second housing type; easy access to the conduit passage area is nonetheless still ensured.

It is likewise within the meaning of the invention if, with respect to an operating position of the system, the cover is mounted on the side or rear or front of the housing of the second housing type. Such mounting of the cover is particularly advantageous if the instillation unit and the vacuum unit are joined together to form a device tower. In this case, mounting the cover on the side, rear or front still ensures the possibility of opening the cover without the instillation unit and the vacuum unit having to be detached from each other.

It is likewise within the meaning of the invention if the cover delimits the conduit passage area of the instillation unit. The interior of the conduit passage area is thus protected by the cover against external influences. Through the possibility of opening the cover, the conduit passage area remains accessible.

One embodiment of the system according to the invention is characterized in that, with respect to an operating position of the system, the first securing mechanism is arranged on an underside of the housing of the first housing type, and the second securing mechanism is arranged on a top of the housing of the second housing type. In this way, the vacuum unit and the instillation unit can be arranged one over the other and can be connected to each other in a fail-safe manner, as a result of which the ease of handling them is improved.

According to a further embodiment of the invention, the housing of the second housing type has at least a third securing mechanism, wherein this third securing mechanism can be connected to a second securing mechanism of a further housing of the second housing type with a form fit, preferably a snap fit, a latching fit, or some other form-fit engagement from behind, or a clamping fit, and wherein the third securing mechanism, with respect to an operating position of the system, is preferably arranged on an underside of the housing of the second housing type. This makes it possible to arrange several instillation units under one vacuum unit. In this way, one or more wounds to be treated can be treated, for example, with several instillation liquids, wherein the different instillation liquids are delivered from different instillation units, thereby permitting, for example, different feed rates and a different composition of the instillation liquids.

The invention likewise relates to a system which comprises a holding device, with a securing mechanism assigned to the holding device, wherein this securing mechanism assigned to the holding device can be connected to the first securing mechanism of the housing of the first type and/or to the third securing mechanism of the housing of the second housing type with a form fit, preferably a snap fit, a latching fit, or some other form-fit engagement from behind, or a clamping fit. In this way, either the vacuum unit can be connected directly to the holding device, or an instillation unit can be secured on the holding device, and, if appropriate, further instillation units are secured on the instillation unit connected to the holding device. A vacuum unit could be arranged above the instillation unit or the instillation units. In this way, a tower composed of instillation units and of a vacuum unit can as it were be held or supported by means of the holding device. By way of the holding device, this tower can then be secured, for example on an infusion stand or a hospital bed, by means of a clamp device arranged on the holding device.

It is particularly advantageous here if the instillation unit or the vacuum unit has a voltage supply interface, which can be connected to a complementary voltage supply interface in the holding device. In this case, the mutually complementary voltage supply interfaces are preferably designed in such a way that a contact between the two interfaces comes about upon connection of the instillation unit to the holding device, such that a supply of voltage to the instillation unit is ensured by the instillation unit being secured on the holding device.

A development of the system according to the invention is characterized in that the instillation unit has a voltage supply interface, which can be connected to a complementary voltage supply interface in the vacuum unit. In this way, the vacuum unit and one or more instillation units can be secured on the holding device, and a voltage supply between the individual units is ensured via the voltage supply interfaces, without wires having to be used for power supply or voltage supply. Advantageously, the interfaces are designed in this case in such a way that a contact for the voltage supply comes about automatically upon connection of the individual units to one another. For example, the instillation unit and the vacuum unit can be designed such that they can be pushed onto each other by means of connection elements like slide seats, wherein the mutually complementary interfaces in this case are designed in such a way that, when the instillation unit and vacuum unit are pushed onto each other, a contact is established between the voltage supply interfaces. The same applies to the design of the interface in the holding device and to the interfaces of the vacuum unit and of the instillation unit that complement the latter interface.

The invention likewise relates to a system in which the conduit passage area is designed for at least two inward conduit portions and at least one outward conduit portion. In this way, inward conduit portions with different instillation liquids can be connected, and these liquids are then delivered separately or jointly to the outward conduit portion.

In a development of the system just described above, the inward conduit portions are brought together in the conduit passage area and open into the outward conduit portion. It is in this way possible, by means of one instillation unit, to combine several instillation liquids and deliver them in a mixed state to a wound. In this way, for example, the use of an antibiotic solution can be combined with the use of an analgesic. Preferably, in this embodiment, flow-controlling or flow-regulating elements are arranged in the area of the conduit passage area and are able to control or regulate the rates at which fluid is fed into the inward conduit portions. Such flow-controlling or flow-regulating elements can be valves, for example, or also clamps.

It is also advantageous if the feed pump, which is preferably designed as a peristaltic pump, is arranged and designed in the area of the outward conduit portion in such a way that, during operation, it exerts a fluid-conveying action on the outward conduit portion. The action of the feed pump on the outward conduit portion makes it possible to operate a plurality of inward conduit portions with only one feed pump. In this way, the size of the instillation unit of the system can be reduced, and costs for the installation of an additional feed pump can be avoided.

It is likewise within the meaning of the invention if the instillation unit has a sensor which is arranged upstream from the feed pump along a fluid delivery direction and is designed to detect when the inward conduit portion contains no instillation liquid. When the instillation liquid to be delivered begins to run out, this is detected by said sensor, and the delivery of the instillation liquid can be stopped and a signal can be output such that additional instillation liquid is made available.

It is likewise within the meaning of the invention if the instillation unit comprises a temperature device which is designed to control and/or regulate the temperature of instillation liquid that is to be delivered. Such a temperature device can be, for example, in the form of a heating element, which is arranged in the area of the outward or inward conduit portion. A temperature sensor is advantageously arranged in this case in the area of the outward conduit portion, said temperature sensor being arranged downstream from the heating element in the delivery direction of the instillation liquid. Such a sensor permits the construction of a control loop, such that the temperature of the instillation liquid to be delivered can be adjusted precisely.

A development of the system according to the invention is characterized in that the cover is designed such that, in the closed state, it delimits the conduit passage area and guides and/or fixes a conduit portion. This can be achieved, for example, by designing the cover such that it has a groove-like recess into which conduit portions arranged in the conduit passage area are introduced when the cover is closed. This prevents the conduit portions from slipping or from being pulled out of the conduit passage area.

It is likewise within the meaning of the invention if the conduit passage area has at least one means which is arranged and designed to fluidically interrupt, in particular to clamp shut, a conduit portion that can be arranged in the conduit passage area. In this way, for example, delivery of the instillation liquid from one inward conduit portion can be briefly interrupted while liquid is delivered from another inward conduit portion. This increases the flexibility of use of the system according to the invention.

The invention also relates to a system comprising at least two instillation units. This has the advantage that two instillation liquids can be delivered to the wound independently of each other. Similarly, the two instillation units can be operated alternately; i.e., as soon as one of the instillation units is unable to deliver any more instillation liquid, a switch-over is made to the other instillation unit, thereby ensuring a seamless supply of instillation liquid to the wound.

A development of the invention is characterized in that the vacuum unit comprises a control unit, and in that signals from the control unit are transmitted to the instillation unit via an interface, in particular a Bluetooth interface or another interface for wireless communication, or an interface for wire-based communication, in particular via a USB interface. In this way, an operation of the system is possible in which the vacuum unit functions as master unit and the instillation unit as slave unit. Preferably, the vacuum unit can operate in this case several instillation units simultaneously. The vacuum unit can in this case have an operating surface, for example in the form of a touch-sensitive screen, via which the entire system, i.e. the vacuum unit and the instillation unit or the instillation units, can be operated.

It is particularly advantageous in this case if the control unit is designed in such a way that it detects an instillation unit connected to the vacuum unit via the interface and adapts the operation of the vacuum unit in such a way that it takes account of an operation of the instillation unit during the operation of the vacuum unit. This means that the application of a vacuum to the wound is suitably adapted depending on whether an instillation unit is or is not operated in parallel with the vacuum treatment. When the wound is treated simultaneously by vacuum and by instillation liquid, it may be advantageous if the feed rate of the vacuum unit is increased, such that the instillation liquid introduced into the wound is removed from the wound via the vacuum unit.

As an alternative to this, it is likewise within the meaning of the invention if the instillation unit comprises a control unit, and signals from the control unit are transmitted to the vacuum unit via an interface, in particular via a Bluetooth interface or another interface for wireless communication, or an interface for wire-based communication, in particular via a USB interface. This embodiment represents the opposite to the embodiment just described above, and therefore the instillation unit now functions as master unit of the system and the vacuum unit functions as slave unit. In this embodiment, it is likewise possible that an instillation unit controls the vacuum unit and also in addition one or more further instillation units.

The operation of the entire system by means of a single master control unit has the advantage that the individual units can be operated in a manner coordinated to each other. There are also advantages in terms of monitoring the course of treatment. Thus, the operating parameters of all the units located in the system can be monitored centrally and, consequently, the operation can be archived.

Also within the meaning of the invention, the control unit is designed in such a way that it detects an optionally present instillation unit or an optionally present vacuum unit via the interface and, in the event of detection, starts a hitherto inactive program routine on the control unit, in particular starts a hitherto inactive program routine which switches a user interface from a first state to a second state, in particular selectively switches hitherto inactive options in the user interface. In this way, for example, options in the user interface of a vacuum unit can remain faded out or deactivated as long as no instillation unit is connected thereto and the vacuum unit is operated on its own. When an instillation unit is connected to the vacuum unit and is detected by the control unit, options in the user interface of the vacuum unit are switched selectively that were previously not selectable or were not even displayed, for example additional selection options or operating options to the two units.

Further features, details and advantages of the invention will become clear from the attached claims, from the drawing, and from the following description of a preferred embodiment of the system according to the invention.

In the drawing:

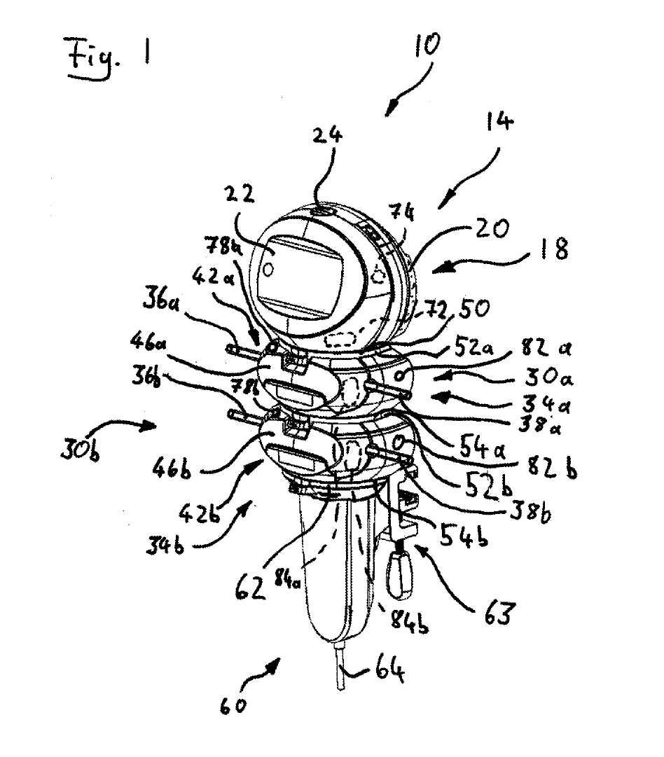

FIG. 1 shows a perspective view of an embodiment of the system according to the invention;

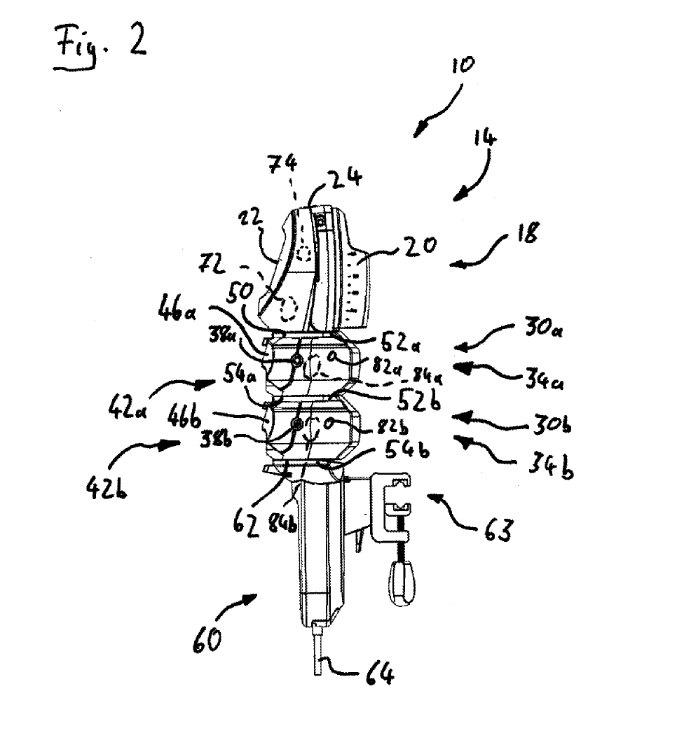

FIG. 2 shows a side view of the system from FIG. 1;

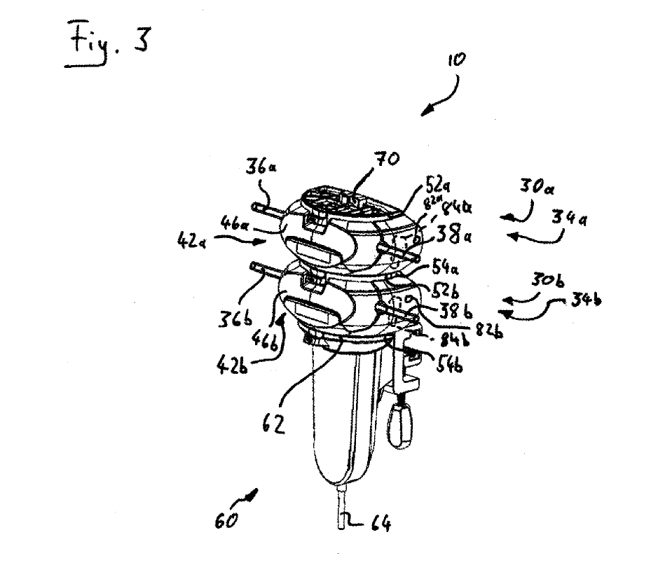

FIG. 3 shows a perspective view of part of the system from FIG. 1;

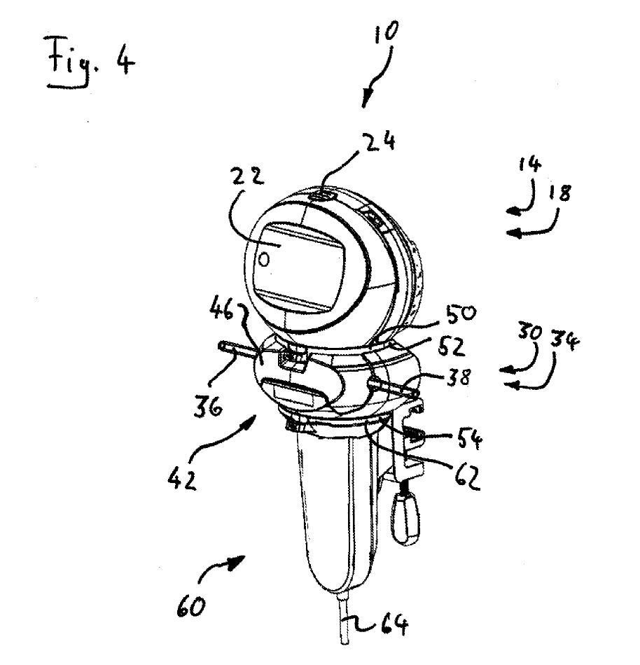

FIG. 4 shows a perspective view of a further embodiment of the system according to the invention;

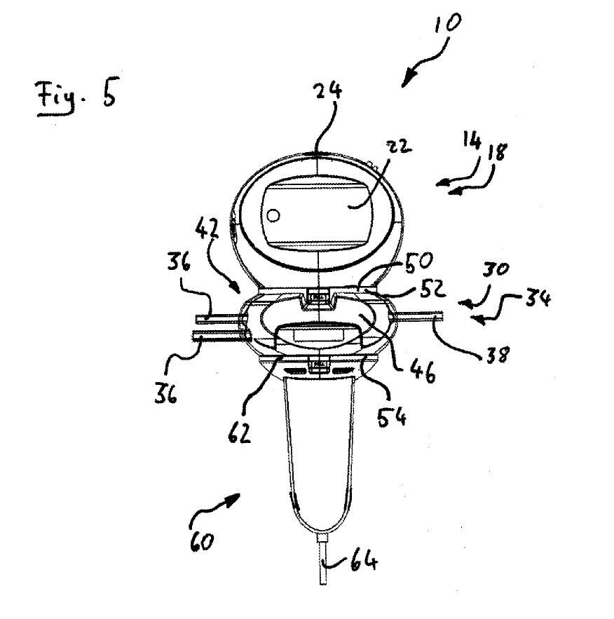

FIG. 5 shows a front view of a further embodiment of the system according to the invention, and

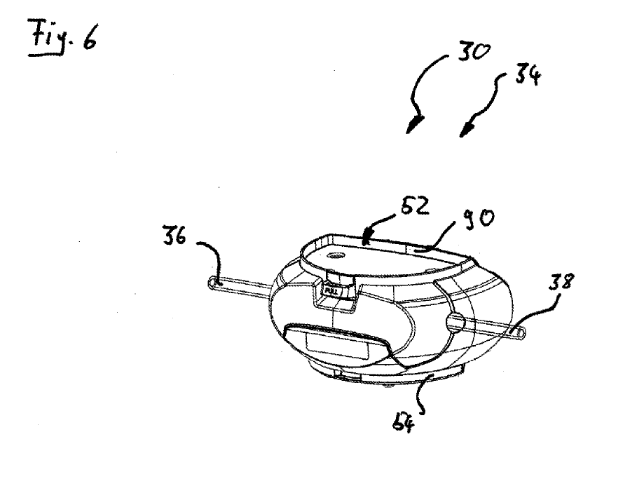

FIG. 6 shows a perspective view of an instillation unit of a further embodiment of the system according to the invention.

FIG. 1 shows a system 10 for combined vacuum and instillation treatment of wounds on the human or animal body. The system 10 comprises a vacuum unit 14 and, for example, two instillation units 30a, 30b. The vacuum unit 14 comprises a housing 18 of a first housing type. A liquid collection container 20 is secured releasably on the housing 18. Opposite the liquid collection container 20, the housing 18 of the first housing type has an operating surface 22. The operating surface 22 is designed as a touch-sensitive screen. The liquid collection container 20 is connected to the housing 18 of the first housing type, for example via a snap-fit connection 24. By actuation of the snap-fit connection 24, the liquid collection container 20 can be released from the housing 18 of the first housing type.

The instillation units 30a, 30b each comprise a housing of a second housing type. They each have an inward conduit portion 36a, 36b and each have an outward conduit portion 38a, 38b. They each comprise a conduit passage area 42a, 42b. The housings 34a, 34b of the second housing type each comprise an openable and closable cover 46a, 46b. The conduit passage areas 42a, 42b of the instillation units 30a, 30b are accessible via the respective covers 46a, 46b and they are preferably delimited by the latter.

The housing 18 of the first type has a first securing mechanism 50. The housings 34a, 34b of the second housing type each have a second securing mechanism 52a, 52b and each have a third securing mechanism 54a, 54b. With respect to the operating position of the system 10 as shown in FIG. 1, the first securing mechanisms 52a, 52b are each mounted on the top of the housings 34a, 34b of the second housing type; the third securing mechanisms 54a, 54b are each mounted on the undersides of the housings 34a, 34b of the second housing type.

The second, upper securing mechanism 52a of the first, upper instillation unit 30a is in this case connected to the first securing mechanism 50 of the vacuum unit 14 by form-fit engagement. The second, upper securing mechanism 52b of the second, lower instillation unit 30b is connected by form-fit engagement to the third, lower securing mechanism 54a of the first, upper instillation unit 30a. The third, lower securing mechanism 54b of the second, lower instillation unit 30b is connected to a holding device 60 via a securing mechanism 62 assigned to this holding device 60. By means of a clamp device 63, the units 14, 30a, 30b connected to each other can be secured, for example, on an infusion stand (not shown) or on a hospital bed.

A line 64 for voltage supply enters the holding device 60. In the area of the securing mechanisms 50, 52a, 52b, 54a, 54b, 62, voltage supply interfaces are in each case provided which come into contact with each other when the units 14, 30a, 30b are connected to the holding device 60.

For example, a control unit 72 of the entire system is arranged in the vacuum unit 14. The control unit 72 is connected to an interface 74 which, in the present illustrative embodiment, is designed for wireless communication. The instillation units 30a, 30b have interfaces 78a, 78b for wireless communication. The instillation units 30a, 30b each have a sensor 82a, 82b, as will be discussed in more detail below. The instillation units 30a, 30b each have a schematically depicted feed pump 84a, 84b, which pumps are designed here as peristaltic pumps.

FIG. 3 shows the system according to the invention from FIGS. 1 and 2, but without the vacuum unit 14. The voltage supply interface assigned to the upper instillation unit 30a can be seen in this view and is designated by reference sign 70. A voltage supply interface matching this interface 70 is provided in the vacuum unit 14.

FIG. 4 shows an embodiment of the system 10 according to the invention with a single instillation unit 30 and a single vacuum unit 14.

The embodiment shown in FIG. 5 differs from the embodiment shown in FIG. 4 in that the instillation unit 30 has two inward conduit portions 36 and one outward conduit portion 38. The inward conduit portions 36 are brought together in the conduit passage area 42 and open into the outward conduit portion 54.

FIG. 6 shows an instillation unit 30 with, for example, one inward conduit portion 36 and, for example, one outward conduit portion 38. The second securing mechanism 52 comprises a border 90. During the connection to a first securing mechanism 50 or a third securing mechanism 54, this border 90 is designed to receive a portion of complementary shape on the securing mechanism.

The instillation unit or instillation units 30, 30a, 30b and the vacuum unit 14 of the respective systems 10 can be connected to each other by means of the securing mechanisms 50, 52a, 52b, 54a, 54b, 62. Advantageously, the securing mechanisms 50, 52a, 52b, 54a, 54b, 62 are in this case designed and arranged in such a way that the instillation units 30, 30a, 30b and the vacuum unit 14 of the systems 10 can be joined together to form a tower, as is shown in FIGS. 1, 2, 4 and 5.

A master control unit 72 is preferably provided in one of the units 14, 30, 30a, 30b and controls the operation of all the units. The control unit 72 determines the operating mode of the vacuum unit 14 and of the instillation units 30, 30a, 30b in this case. In the embodiment shown in FIGS. 1 and 2, the control unit 72 transmits data concerning the operating mode to the interfaces 78a, 78b of the instillation units 30a, 30b via the interface 74 assigned to the vacuum unit 14.

The sensors 82a, 82b provided in the instillation units 30a, 30b detect when no instillation liquid is present in the inward conduit portions 36a, 36b. If this is the case, the operation of the respective peristaltic pump 84a, 84b can be stopped, and a signal can be output which requests the provision of further instillation liquid.

1-22. (canceled)

23. A system for combined vacuum and instillation treatment of wounds on a human or animal body, the system comprising:
a vacuum unit having a housing of a first housing type, said housing of said first housing type having a first securing mechanism, said vacuum unit further comprising a pump for providing a vacuum as well as a liquid collection container for receiving bodily fluids or for receiving wound secretions aspirated from a wound, wherein said liquid collection container is releasably secured on said housing of said first housing type; and
an instillation unit having a housing of a second housing type, said instillation unit also having a conduit passage area for at least one inward conduit portion and for an outward conduit portion and a feed pump or a peristaltic pump, said housing of said second housing type having an openable and closable cover through which said conduit passage area is accessible, wherein said housing of the second housing type further comprises a second securing mechanism structured for connection to said first securing mechanism with a form fit, a snap fit, a latching fit, a form-fit engagement from behind or a clamping fit, said housings of said first and said second housing types thereby being attachable to and separable from each other.

24. The system of claim 23, wherein said cover is mounted pivotably on said housing of said second housing type.

25. The system of claim 23, wherein, with respect to an operating position of the system, said cover is mounted on a side, rear or front of said housing of said second housing type.

26. The system of claim 23, wherein said cover delimits said conduit passage area of said instillation unit.

27. The system of claim 23, wherein, with respect to an operating position of the system, said first securing mechanism is arranged on an underside of said housing of said first housing type and said second securing mechanism is arranged on a top of said housing of said second housing type.

28. The system of claim 23, wherein said housing of said second housing type has at least a third securing mechanism, wherein said third securing mechanism is structured for connection to a fourth securing mechanism of a further housing of said second housing type with a form fit, a snap fit, a latching fit, a form-fit engagement from behind or a clamping fit, wherein, with respect to an operating position of the system, said third securing mechanism is disposed on an underside of said housing of said second housing type.

29. The system of claim 28, further comprising a holding device having a securing mechanism, wherein said securing mechanism of said holding device is structured for connection to said first securing mechanism of said housing of said first housing type and/or to said third securing mechanism of said housing of said second housing type with a form fit, a snap fit, a latching fit, a form-fit engagement from behind or a clamping fit.

30. The system of claim 29, wherein said instillation unit has a voltage supply interface, which is structured for connection to a complementary voltage supply interface in said holding device.

31. The system of claim 23, wherein said instillation unit has a voltage supply interface, which is structured for connection to a complementary voltage supply interface in said vacuum unit.

32. The system of claim 23, wherein said conduit passage area is designed for at least two inward conduit portions and at least one outward conduit portion.

33. The system of claim 32, wherein said inward conduit portions are brought together in said conduit passage area and open into said outward conduit portion.

34. The system of claim 32, wherein said feed pump is arranged in an area of said outward conduit portion and designed in such a way that, during operation, said feed pump exerts a fluid-conveying action on said outward conduit portion.

35. The system of claim 23, wherein said instillation unit has a sensor which is arranged upstream of said feed pump along a fluid delivery direction and is designed to detect if said inward conduit portion contains no instillation liquid.

36. The system of claim 23, wherein said instillation unit comprises a temperature device which is designed to control and/or regulate a temperature of instillation liquid that is to be delivered.

37. The system of claim 23, wherein said cover is designed such that, in a closed state, said cover delimits said conduit passage area and guides and/or fixes a conduit portion.

38. The system of claim 23, wherein said conduit passage area has at least one mechanism which is arranged and designed to fluidically interrupt or to clamp shut a conduit portion that is structured to be arranged in said conduit passage area.

39. The system of claim 23, wherein the system comprises at least two instillation units.

40. The system of claim 23, wherein said vacuum unit comprises a control unit, signals from said control unit being transmitted to said instillation unit via an interface, via a Bluetooth interface, via an interface for wireless communication, via an interface for wire-based communication or via a USB interface.

41. The system of claim 40, wherein said control unit is designed to detect an instillation unit connected to said vacuum unit via said interface and adapts an operation of said vacuum unit in such a way as to take account of an operation of said instillation unit and/or adapts an operation of said instillation unit in such a way as to take account of an operation of said vacuum unit.

42. The system of claim 23, wherein said instillation unit comprises a control unit, signals from said control unit being transmitted to said vacuum unit via an interface, via a Bluetooth interface, via an interface for wireless communication, via an interface for wire-based communication or via a USB interface.

43. The system of claim 42, wherein said control unit is designed to detect a vacuum unit connected to said instillation unit via said interface and adapts an operation of said vacuum unit in such a way as to take account of an operation of said instillation unit and/or adapts an operation of said instillation unit in such a way as to take account of an operation of said vacuum unit.

44. The system of claim 40, wherein said control unit is designed to detect an instillation unit or a vacuum unit via said interface and, in the event of detection, starts a hitherto inactive program routine on said control unit or starts a hitherto inactive program routine which switches a user interface from a first state to a second state or which selectively switches hitherto inactive options in said user interface.
